(19) United States
(12) Patent Application Publication
(10) Pub. No.: US 2012/0141618 A1
(43) Pub. Date: Jun. 7, 2012

(54) PROCESS FOR PREPARING INTEGRAL OLIVE JUICE, USING THIS PROCESS TO OBTAIN THE COMPOSITION AND ITS APPLICATION IN THE FIELD OF COSMETICS AND NUTRITION

(76) Inventors: Raphaël Colicci, Saint-Privat (FR); Jacques Dubois, Narbonne (FR); Jean-Louis Lafaurie, Herepian (FR)

(21) Appl. No.: 13/389,807

(22) PCT Filed: Aug. 10, 2010
(86) PCT No.: PCT/FR2010/051688

§ 371 Date: Feb. 10, 2012

(30) Foreign Application Priority Data
Aug. 11, 2009 (FR) 0955618

(51) Int. Cl. A61K 36/63; A23D 7/00; A61P 35/00; A61P 39/06; A61K 8/97; A61P 9/00

(52) U.S. Cl. 424/777; 426/602

(57) ABSTRACT

This invention concerns a process for preparing integral olive liquor consisting of the following stages:

a) selecting the olives according to their maturity,
b) grinding and mixing the olives selected in the form of drupes,
c) pressing the paste obtained in the previous phase,
d) separating the liquor obtained in order to separate it from the solid parts,
e) collecting the integral olive liquor in the form of a stable, gelified emulsion.

A composition based on integral olive liquor obtained according to the process subject of the invention, comprising a mixture of the oily phase and the aqueous phase of the olive is also claimed in addition to its use in the field of cosmetics and/or nutrition.

The purpose of this invention concerns a procedure for preparing integral olive juice in addition to a composition based on integral olive juice obtained using this process. Using this composition in the field of cosmetics and nutrition is also the subject of this invention.

The olive tree (Olea europea) and its by-products have been well-known for millennia. Traces of fossilised leaves dating back to 37 000 BC can be found on the Santorin site, and traces of coal and olive tree pollen dating back to 12,000 BC have been identified at the edge of the Sahara.

The olive is a drupe with a hard kernel growing on trees which are sometimes hundreds of years and even thousands of years old in Palestine, Greece and Sicily. Numerous varieties of olive trees are grown around the Mediterranean and also in California, Australia, South Africa and China. Green olives are gathered before the fruit is completely ripe (in September for the Mediterranean basin), whereas the black colour only develops when the fruit is completely ripe (from December to February for the Mediterranean basin).

As a result of the bitterness caused by the main heteroglucoside (oleuropein) they contain in their pulp, the olives are unsuitable for consumption until they are treated with an alkaline solution which causes this bitter glucoside to hydrolyse; after rinsing in water they are kept in brine.

The oil is obtained using various well-known processes: olives intended for this production are washed, then ground into a paste; after mixing this paste is pressurized then decanted or centrifuged to separate the solid elements, or “cakes”, from the liquid phase consisting of oil and an aqueous solution known as “olive water”. The oil produced varies according to the species, the extraction process and on how ripe the olive is. That is why, with olives which are completely ripe, 12 to 25 kg of oil for 100 kg of fruit can often be obtained.

We must be aware that this output is much lower with olives gathered before maturity and drops to 5-6% of oil in cases of early harvesting. Traditionally the oil which has a special colour and composition called “omphacin” was used by Greek perfumiers then by the Romans as body unction.

Very soon humans knew how to use the olive and its by-products in various ways. For dietary purposes, the green or black fruit are suitable for a wide range of preparations and constitute highly appreciated local specialties.

In addition, the oil preferably obtained by cold pressure (extra virgin oil) is the essential element of the Cretan diet and more widely the Mediterranean. The paradox of this diet, which is now clearly understood, is associated with the composition of the triglycerides 75% of which include monounsaturated fatty acid: oleic acid. In addition, we discovered the presence of phenolic compounds: heteroglucosides (oleuropein, ligstroside or oleocanthal) and the phenols which are a by-product of their hydrolysis (tyrosol, hydroxytyrosol). We should be aware that olive oil also contains vitamins (especially A and E), squalane and sterols in the unsaponifiable fraction.

Together with the dietary applications, the oil is used in the pharmaceutical field by mouth or for washing out because of its cholagogical, slightly laxative properties.

For external use, its softening and emollient properties justify its use in dermatology (particularly oleo-calcareous); it is used as a vehicle in various Codex preparations: camphorated oil or niaouli essential oil for example. It is used as a solvent for certain injectable preparations after being neutralised and sterilised in conditions defined by the pharmacopeia.

In the industrial field, olive oil is used in important applications, such as manufacturing soaps or for preparing cosmetology products.

Cakes or “grignons”, the solid residue separated after mixing drupes is recovered and may be used to feed cattle or even as fertiliser.

Finally, the olive water, obtained after systematically separating the oil from the liquid phase, is an effluent rich in organic substances, creating a real pollution problem to the oil industry, in the absence of any way of making its component parts more valuable.

Currently, since the advantages of the Cretan diet have become obvious, a brand new outlet for using the olive for dietary and therapeutic purposes has been developed. Recent scientific research has revealed the effects of polyphenols on olive oil: its antioxidant action and ability to increase levels of HDL cholesterol, thus explaining why phenomena associated with ageing slow down, preventing certain diseases such as cardiovascular disease, osteoporosis, tumour formation.

This invention aims to use the well-known properties of oil resulting from its composition (triglycerides of monounsaturated fatty acids, heteroglucosides, vitamins A and E, squalane, sterols, phenol compounds) and olive water rich in advantageous active ingredients, particularly mineral salts (Ca, Mg, P, K) and polyphenols at high concentration, as yet unused. However these constituents are constantly eliminated to obtain oil which is used in isolation because of its intrinsic properties. So this olive water constitutes a superfluous effluent, creating just as much pollution since modern industrial continual extraction techniques result in a considerable increase in their volume because of the addition of extra water during centrifuging processes aimed at increasing oil extraction output.

As for the traditional process for obtaining oil, this employs a discontinuous method comprising stages of grinding, mixing then pressing and finally decantation and centrifugation, without adding water. The aqueous phase (olive water) is then systematically separated from the oil phase which is considered to be the best product of the fruit. Using this traditional process, the olive water is more concentrated (content in dry extract approximately twice as high) than that obtained using an industrial process, thus constituting waste whose polluting effect has not been considered advantageous for a long time.

Tests to valorise this olive water have been performed in order to reduce the polluting nature of this residue and to use some of its constituents, but these have proved unsuccessful to date. The difficulty lies in the instability of the polyphenols which rapidly oxidise, preserving this problematic aqueous phase. Some people believed that this problem could be resolved by spraying or evaporating this solution in order to obtain a stable powder, more easy to handle. However this conversion in itself causes rapid degradation of the very compounds it aims to stabilise.

The advantages resulting from combining the stabilised active ingredients with olive oil have never been realized. FR 2867071 describes a composition combining a dry extract of olive water with olive oil peroxidised by ozonisation to give new properties to this phase. The product obtained then becomes an item requiring complex and costly processes, burdening the manufacturing process and also adding cost to a converted product which no longer has the natural characteristics of olive oil and no longer possesses the same qualities.

This invention provides a solution to these problems. Taking inspiration from the age-old method which is no longer used, the inventers have refined a process which can directly obtain, without any separation of the aqueous and oily phases, integral olive liquor, after pressing the paste and separating the solid residues while obtaining good stabilisation of the active ingredients of the aqueous phase.

More specifically, this invention consists of a process for preparing integral olive liquor characterised in that it consists of the following stages:

a) selecting the olives according to how ripe they are,

b) grinding and mixing the olives selected in the form of drupes,

c) pressing the paste obtained in the previous phase,

d) separating the liquor obtained in order to separate it from the solid parts,

e) collecting the integral olive liquor in the form of a stable, gelified emulsion.

Unlike the age-old method, no longer used, whose aim is to separate the two phases in order to make better use of the intrinsic properties of olive oil and at the same time eliminate the olive water which is considered to be harmful, the process according to the invention specifically aims to keep the two phases in close contact. Quite unexpectedly, the inventers actually observed that, in addition to the advantage in terms of simplicity and preparation time, the quality and properties of the oil phase are not damaged by the presence of olive water. On the other hand, the benefits of olive oil are potentialised by this association as a result of the constituents and active ingredients of the aqueous phase. These active ingredients show that they are even more beneficial if they are in their natural form, and are not converted by an additional treatment phase. Contact with the oily phase also seems to have a protective and stabilising action in relation to the constituents of the olive water whose properties remain optimum.

The integral olive liquor obtained at the end of this process takes the form of a gelified emulsion naturally stabilised by the spontaneous interactions between the oily phase and the aqueous phase. To increase the time of preservation and stability of this integral liquor, it is possible to incorporate, immediately after stage e) viscosing agents which may also be emulsifying agents or a mixture of these.

In order to optimise the quality of the integral olive liquor, the process subject of the invention is preferably carried out cold in a sheltered place.

In the course of the work to carry this out and to refine this new process, the inventers had to use olives obtained throughout the possible harvest time, ranging respectively from a state of early to advanced ripening (from September to January-February for the Mediterranean basin). This chance event allowed them to discover the existence of important variations in the composition between the products obtained when the olives were ripe and those obtained from fruit gathered as early as possible, similar to those processed in antiquity to obtain the oil omphacin. These variations mainly take the form of colour, vitamin activity, quantity of oleic acid and biophenols, as well as in terms of output in liquor and oil. This data has been measured and appears in the examples in this application.

So, the process according to this invention may include in stage a) the selection of olives mainly gathered at an early stage of maturity (characterised by green olives, firm and smooth drupes, rich in water) which means that all the advantageous properties associated with omphacin can be used. In this case, by preference, stage a) includes the selection of olives solely gathered at an early stage of maturity.

As an alternative, the process according to the invention can also comprise, at stage a), the selection of olives mainly gathered at an advanced stage of maturity (characterised by black olives because of a higher content of verbascoside, ripe and wrinkled drupes, whose water content is lower and the oil content higher) in order to benefit from the advantageous properties of a riper oil.

In this case, by preference, stage a) includes selecting olives solely gathered at an early stage of maturity.

This invention also concerns a composition based on integral olive liquor obtained according to the process described above, characterised in that it comprises a mixture of the oily phase and the aqueous olive liquor unadulterated by the aggressive stages of treatment. This integral olive liquor actually contains all the ingredients of an olive at the highest possible concentration and in the most natural form, which is why the Cretan diet is advantageous (oily glycerides, phenol compounds, vitamins A and E, sterols, squalanes, etc.), with a view to using them nutritionally. It also has all the qualities required for acting as a basis for cosmetic preparations.

Depending on when the fruit is gathered, the integral olive liquor takes the form of a mixture consisting of an oil whose proportion varies between 5 and 25% by weight, and an aqueous solution consisting of between 95 and 75% by weight. The composition according to the invention thus preferably consists of 5 to 25% by weight of this composition in the oily phase, with 95 to 75% by weight of this composition in the aqueous phase.

The possibility of adjusting the relative percentage of the oily phase, by adding virgin oil obtained independently of the process claimed is not excluded, in order to adjust the proportion of oil present in the finished product according to its application. This addition of oil is able to reinforce the nourishing and protective qualities of a night cream for example. This is not required if the wish is to obtain an emulsion whose texture is lighter than a hydrating milk.

In order to facilitate its use, and preservation, this new product can be stabilised by using various additives which preferably comply with the requirements of bio labels (such as ECOCERT in France, for example). In one particular production method, the composition according to the invention may also include additives selected from among the gelling agents, hydrosoluble antioxidants, preservatives, chelators or a mixture of these.

Adding gelling agents, for example, is able to increase viscosity and maintain the homogeneous mixture without separating the two phases. For information and without being comprehensive, we can list the traditional gelling agents, selecting those which are recommended for use in food: alginates, carrageenans, pectins, arabic gum, adragant gum and especially guar gum and xanthan gum. Their concentration varies between 0.1 and 3% by weight, depending on the product.

It is possible to add hydrosoluble antioxidants, preferably natural, such as ascorbic acid.

The microbiological protection may be obtained from a physical process such as traditional UHT processing for milk or by adding authorised preservatives: salicylic acid, benzoic acid, sorbic acid as well as their salts or a combination of these. The concentration is between 0.05 and 1% by weight.

Chelators may also be incorporated in the end composition.

By chance, the inventers also considered using the composition obtained according to the process subject of the invention, without any other treatment, in the field of cosmetics and/or nutrition.

The applications envisaged are actually used directly of this integral liquor, eliminating the separation of the aqueous and oily phases. The product obtained prove to be particularly advantageous as it contains all the liquid constituents, soluble in water and oil, without losing any active ingredients in the natural state, without any risk of damage, cutting down on processes, by reducing the energy used and avoiding the accumulation of effluents which are undesirable in the climate of ecological concern.

In the case of a composition produced mainly with olives gathered at an early stage of maturity the composition obtained using the process subject of the invention is preferably carried out in the field of cosmetics. This use has proved particularly advantageous through the anti-radical, anti-inflammatory, non therapeutic, emollient and softening effect of this composition.

As an alternative, the composition obtained using the process subject of the invention is employed in the field of nutrition with regard to a composition produced mainly with olives gathered at a stage of advanced maturity. This use has proved particularly advantageous as a result of the anti-radical, cardiovascular disease prevention and tumoral lesion prevention action of this composition.

Prepared in this way, this new product is packed in sealed, hermetically sealed containers, completely filled and stored away from light and heat, with it being possible to replace the remaining air with carbon dioxide or nitrogen; their volume is adapted to that of the final operating unit.

The following examples illustrate the invention in order to show the characteristics and the advantages more clearly, however without reducing its impact in any way.

### EXAMPLES

In order to illustrate the differences in composition between early olives and ripe olives, certain characteristics have been measured with olives from the “Picholine” and “Lucques” variety gathered in September (omphacin) or in December (traditional oil) 2008.

Colour:

_{{0>}Le spectre d'absorption révèle un pic à 420 nm dans l'omphacine et l'huile classique; un deuxième pic à 670 nm est observé dans l'omphacine seule, —activité vitaminique E: _{<}0{>}The spectrum of absorption reveals a peak at 420 nm in omphacin and traditional oil; a second peak at 670 nm is observed in omphacin alone.

Vitamin E Activity:_{<0}}
36 mg for 100 grams of fat (GFM) in omphacin from Picholine, compared with 17 mg/100 GFM in Picholine traditional oil.
14.5 mg/100 GFM of Lucques omphacin, compared with 8,1 mg/100 GMG of traditional Lucques oil. i.e. a tocotrienol content approximately twice as high in omphacin when compared with that from traditional oil.

Oleic Acid:
81,5 g/100 g of omphacin from Picholine, compared with 78,6 g/100 g of traditional Picholine oil.
77.4 g/100 g of omphacin from Picholine, compared with 75.4 g/100 g of traditional Lucques oil.
   i.e. a higher content of oleic acid in omphacin compared with that from traditional oil.

Total Biophenols:
225 mg/kg of omphacin from Picholine (of which 221 mg/kg are in a complex form), compared with 160 mg/kg from traditional Picholine (of which 134 mg/kg in a complex form).
211 mg/kg of omphacin from Lucques (of which 205 mg/kg are in a complex form), compared with 123 mg/kg from traditional Picholine (of which 100.4 mg/kg is in a complex form), i.e. a higher content of oleic acid in omphacin than in traditional oil.

| output in liquor and oil: | early olives | ripe olives |
|---|---|---|
| Oil | 5-9% | 12-25% |
| Olive water | 95-91% | 88-75% |

COMPOSITION 1: Stabilised Integral Olive Liquor

Liquor from 100 g of ripe olives (Picholine)

_{{0>}-additifs:_{<}0{>}-additives:_{<}0{>}gomme xanthane 0,4 g_{<}0{>}0.4 g xanthan gum<_{0}}
_{{0>}ascorbate de sodium 0,3 g<_{}0{>}0.3 g sodium ascorbate _{<0}}
_{{0>}sorbate de potassium 0,5 g<_{}0{>}0,5 g potassium sorbate

The ingredients are mixed avoiding any incorporation of air, under cold conditions.

The composition may then undergo UHT processing and be packaged in a sterile environment.

COMPOSITION 2: Stabilised Integral and Olive Liquor

Liquor from 100g of early olives (Picholine)

Additives: 0.5 g guar gum
0.4 g sodium ascorbate
0.25 g potassium sorbate
0.25 g sodium benzoate

Same Operational Mode as for Composition 1

The composition according to this invention may present in various forms of end products, with it being possible to combine the active ingredients, i.e. the integral liquor, with a multitude of compounds which are themselves either neutral or equipped with advantageous compounds (oil, water, etc).

Cold manufacture is obviously preferred as it preserves all the properties and benefits of integral olive liquor. It may be obtained by emulsifying the ingredients at any convenient time.

In the form of seasoning or vinaigrette, integral liquor from ripe olives (20% by weight) may be combined with citric acid, aromatics, salt and pepper. A light recipe may contain 7% by weight of integral liquor from green olives instead of 20% of integral liquor from ripe olives.

Cosmetic milks based on integral liquor from green olives, day creams based on integral liquor from ripe olives or night creams based on integral liquor from ripe olives containing 10% by weight of virgin olive oil may also be obtained in addition to all presentations intended for use on the body or hair formulated from integral liquor from green or ripe olives: in particular bath products, body oils, either in the form of a clay or mud pack, and non-rinse haircare products. These products are formulated according to the rules that any professional person knows how to define, incorporating. In particular, various preservatives (benzyl alcohol, docosahexaenoic acid or DHA, etc.), emulsifiers (olivem, oleate, stearate, eumulgen VL75, etc.), essential citrus oils, for example, as well as colorants (TiO2) in proportions that any professional person may adapt according to the envisaged applications.

1. Process for preparing integral olive liquor characterised in that it consists of the following stages:
a) selecting the olives according to their maturity,
b) grinding and mixing the olives selected in the form of drupes,
c) pressing the paste obtained in the previous phase,
d) separating the liquor obtained in order to separate it from the solid parts,
e) collecting the integral olive liquor in the form of a stable, gelified emulsion.

2. Process according to claim 1, characterised in that it comprises the addition immediately after stage e) of viscosing agents which may also be emulsifiers or a mixture of these.

3. Process according to claim 1, characterised in that at stage a) it includes the selection of olives mainly gathered at an early stage of maturity.

4. Process according to claim 3, characterised in that at stage a) it includes the selection of olives mainly gathered at an early stage of maturity.

5. Process according to claim 1, characterised in that at stage a) it includes the selection of olives mainly gathered at an advanced stage of maturity.

6. Process according to claim 5, characterised in that at stage a) it includes the selection of olives mainly gathered at an advanced stage of maturity.

7. Composition based on integral olive liquor obtained using claim 1, characterised in that it comprises a mixture of the oily phase and the aqueous phase of olive.

8. Composition according to claim 7, characterised in that the invention preferably consists of 5 to 25% by weight of said composition of the oily phase with 95 to 75% by weight of said composition of the aqueous phase.

9. Composition according to claim 7, characterised in that it includes additives selected from among the gelling agents, hydrosoluble antioxidants, preservatives, chelators or a mixture of these.

10. Use of a composition according to claim 7 in the field of cosmetics and/or nutrition.

11. Use according to claim 10, characterised in that it is preferably carried out in the field of cosmetics in the case of a composition produced mainly with olives gathered at an early stage of maturity.

12. Use according to claim 11, characterised in that it is able to offer an anti-radical, emollient and softening action.

13. Use according to claim 10, characterised in that it is preferably carried out in the field of nutrition in the case of a composition produced mainly with olives gathered at an advanced stage of maturity.

14. Use according to claim 13, characterised in that it is able to offer an antiradical action, to prevent cardiovascular disease and tumoral lesions.

15. Process according to claim 2, characterised in that at stage a) it includes the selection of olives mainly gathered at an early stage of maturity.

16. Process according to claim 2, characterised in that at stage a) it includes the selection of olives mainly gathered at an advanced stage of maturity.

17. Composition based on integral olive liquor obtained using claim 2, characterised in that it comprises a mixture of the oily phase and the aqueous phase of olive.

18. Composition based on integral olive liquor obtained using claim 3, characterised in that it comprises a mixture of the oily phase and the aqueous phase of olive.

19. Composition based on integral olive liquor obtained using claim 4, characterised in that it comprises a mixture of the oily phase and the aqueous phase of olive.

20. Composition based on integral olive liquor obtained using claim 5, characterised in that it comprises a mixture of the oily phase and the aqueous phase of olive.
